(19) United States
(12) Patent Application Publication
(10) Pub. No.: US 2013/0116190 A1
(43) Pub. Date: May 9, 2013

(54) HYALURONIC ACID-COLLAGEN MATRICES FOR TISSUE ENGINEERING

(71) Applicant: ALLERGAN, INC., Irvine, CA (US)

(72) Inventors: Jacob Pollock, Charleston, WV (US); Xiaojie Yu, Irvine, CA (US); Christopher Traina, Goleta, CA (US); Nicholas J. Manesis, Summerland, CA (US); Lauren Kokai, Santa Barbara, CA (US); Dennis Van Epps, Goleta, CA (US); Darin Messina, Santa Barbara, CA (US)

(73) Assignee: ALLERGAN, INC., Irvine, CA (US)

(21) Appl. No.: 13/728,855

(22) Filed: Dec. 27, 2012

Related U.S. Application Data
(63) Continuation-in-part of application No. 13/667,581, filed on Nov. 2, 2012.
Application No. 13/667,581 is a continuation-in-part of application No. 13/605,565, filed on Sep. 6, 2012.
Application No. 13/605,565 is a continuation-in-part of application No. 13/603,213, filed on Sep. 4, 2012.
(60) Provisional application No. 61/580,971, filed on Dec. 28, 2011.
(60) Provisional application No. 61/555,970, filed on Nov. 4, 2011.
(60) Provisional application No. 61/531,533, filed on Sep. 6, 2011.
(60) Provisional application No. 61/531,533, filed on Sep. 6, 2011.

(51) Int. Cl. A61K 38/39

(52) U.S. Cl. CPC A61K 38/39
USPC 514/17.2

(57) ABSTRACT

Hydrogels comprising a macromolecular matrix including a crosslinked combination of hyaluronic acid and collagen, are provided for tissue engineering applications.

This application claims priority to U.S. Provisional Patent Application No. 61/580,971, filed Dec. 28, 2011, and is a continuation-in-part of U.S. patent application Ser. No. 13/667,581, filed on Nov. 2, 2012, which claims priority to U.S. Provisional Patent Application No. 61/555,970, filed Nov. 4, 2011, and which is also a continuation-in-part of U.S. patent application Ser. No. 13/605,565, filed on Sep. 6, 2012, which claims priority to U.S. Provisional Patent Application No. 61/531,533, filed Sep. 6, 2011, and which is also a continuation-in-part of U.S. patent application Ser. No. 13/603,213 filed on Sep. 4, 2012, which claims priority to U.S. Provisional Patent No. 61/531,533, filed Sep. 6, 2011, the entire content of each of these documents being incorporated herein by this reference.

This application generally relates to biocompatible, implantable compositions and more specifically relates to hyaluronic acid-collagen based compositions useful for tissue support, healing and repair, for example, deep tissue support, healing and repair.

Tissue engineering is a newly emerging biomedical technology that utilizes biomaterials which assist and/or accelerate the regeneration and repair of defective and/or damaged tissues based in part on the natural healing potentials of patients themselves. Ideally, biomaterials for tissue engineering provide an environment that enhances and/or regulates cell growth and proliferation. For example, biomaterial scaffolds have been investigated to enhance the proliferation and differentiation of cell potential for tissue regeneration.

Tissue engineering is a significant potential alternative or complementary approach to conventional surgical options for repair of damaged or failing tissues or organs. An increasing number of materials, both synthetic and natural types, are now being engineered specifically for these purposes.

It has been a challenge to develop safe and effective biomaterials, for example, implantable or injectable biocompatible materials which can, upon introduction into a patient, will promote long term structural support, healing, repair, and regeneration of defective, damaged or failing tissue.

### SUMMARY

The present invention generally relates to hydrogel compositions and methods useful for treating tissue, for example, augmenting, supporting, promoting repair and/or regeneration of soft tissue, including tendon, muscle and organs, in a patient.

In one aspect, a product is provided comprising hydrogel compositions in the form of an injectable scaffold suitable for augmenting tissue, filling in tissue voids and providing structural support to damaged tissue.

In another aspect, methods are provided for promoting tissue generation and tissue repair in a patient, the methods generally comprising introducing into a patient a hydrogel composition.

Further included are hydrogel compositions useful for treating a human or veterinary subject having a disorder characterized by tissue damage or loss. In addition, methods of treating soft tissue in a patient comprising implanting a hydrogel composition into a soft tissue of the human being to thereby improve, heal or repair the soft tissue.

Generally, the hydrogel compositions comprise a hyaluronic acid (HA) component, a collagen component and a crosslinking component. The hyaluronic acid component and the collagen component are crosslinked to one another by an ester bond or an amide bond.

In an exemplary embodiment, the collagen component is at least one of collagen type I or collagen type III.

In one aspect of the invention, the compositions have a weight ratio of the hyaluronic acid component to the collagen component of about 0.5 to about 7. Stated another way, the composition has a weight ratio of the hyaluronic acid component to the collagen component of about 0.5:1 to about 7:1.

For example, the weight ratio of the hyaluronic acid component to the collagen component may be about 1, about 2, about 3, about 4, about 5, about 6, or about 7.

In some embodiments, the composition has a weight ratio of the hyaluronic acid component to the collagen component of about 1 to about 3. In other words, in some particular embodiments, the composition has a weight ratio of the hyaluronic acid component to the collagen component of about 1:1 to about 3:1.

In some embodiments, the collagen concentration is at least about 3 mg/ml up to about 12 mg/ml and the hyaluronic acid concentration is about 3 mg/mL to about 20 mg/mL.

In some embodiments, the collagen concentration is about 3 mg/mL, and the hyaluronic acid concentration is about 3 mg/ml to about 24 mg/mL. For example, in some embodiments, the collagen concentration is about 3 mg/mL, and the hyaluronic acid concentration is about 3 mg/mL, or about 6 mg/mL, or about 10 mg/mL, or about 12 mg/ml, or about 16 mg/ml, or about 18 mg/ml, or about 20 mg/ml, or about 22 mg/mL, or about 24 mg/mL.

In one embodiment, the collagen concentration is about 6 mg/mL, and the hyaluronic acid concentration is about 12 mg/mL.

In another embodiment, the collagen concentration is about 8 mg/mL, and the hyaluronic acid is about 16 mg/mL.

In yet another embodiment, the collagen concentration is about 12 mg/mL, and the hyaluronic acid concentration is about 12 mg/mL.

In a further embodiment, the collagen concentration is about 12 mg/mL and the hyaluronic acid concentration is about 16 mg/mL.

The compositions may comprise a crosslinked macromolecular matrix comprising a hyaluronic acid component, a collagen component and a crosslinking component. At least a portion of the crosslink units comprise an ester bond or an amide bond.

Methods for making such hydrogel compositions useful for treating soft tissue are also provided. The methods generally comprise crosslinking hyaluronic acid and collagen by dissolving a hyaluronic acid and a collagen in an aqueous solution to form an aqueous pre-reaction solution, wherein the aqueous pre-reaction solution further comprises a salt or has a low pH; and modifying the aqueous pre-reaction solution to form a crosslinking reaction mixture comprising: the hyaluronic acid; the collagen; a water soluble coupling agent; and the salt; and wherein the crosslinking reaction has a higher pH than the aqueous pre-reaction solution; and allowing the crosslinking reaction mixture to react to thereby crosslink the hyaluronic acid and the collagen.

### BRIEF DESCRIPTION OF THE DRAWINGS

Aspects and advantages of the present disclosure may be more clearly understood and/or better appreciated with reference to the following Detailed Description, when considered in conjunction with the accompanying drawings of which:

FIGS. 1A and 1B show plots of frequency sweep and strain sweep, respectively, for a hydrogel composition in accordance with this disclosure;

FIG. 2 is an extrusion profile through a 30 G needle for a hydrogel composition of this disclosure.

FIGS. 3A-3C show, respectively, micrographs (at 5× magnification) of (A) tissue adjacent to an implanted control composition of commercial crosslinked hyaluronic acid gel, (B) tissue adjacent to an implanted composition of Example 3, and (C) tissue adjacent to an implanted composition from Example 4.

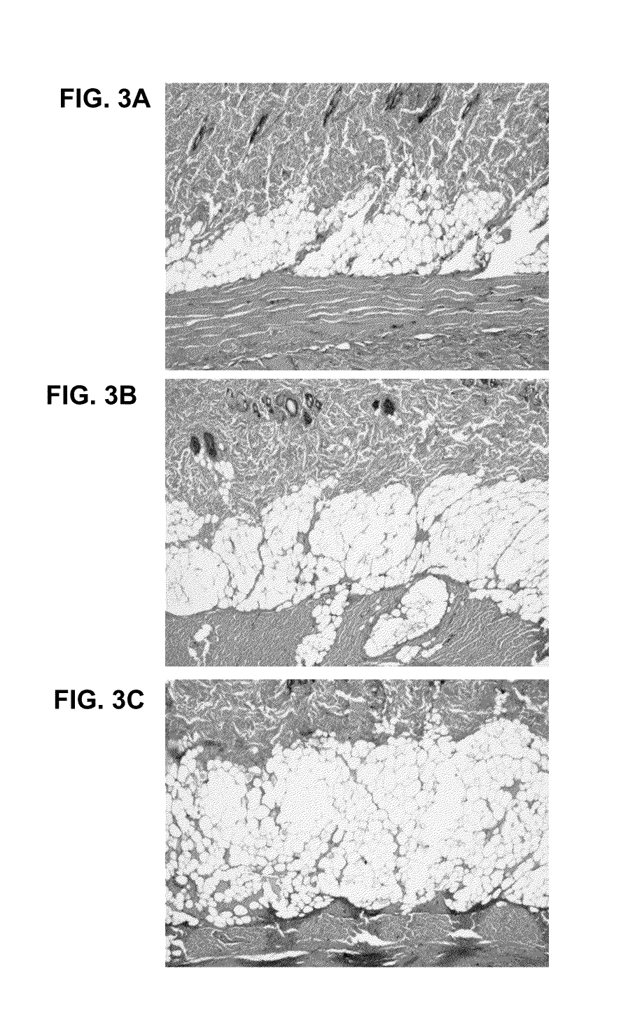

FIG. 4 is a chart of photographs showing explanted hydrogel compositions showing different amounts of new vessel ingrowth.

### DETAILED DESCRIPTION

Methods of treating soft tissue are provided. Such a method may comprise introducing a hydrogel composition into a soft tissue of a patient, thereby treating a soft tissue condition.

The composition may be in any form suitable for introducing into tissue, for example, mammalian tissue, for example human tissue, to treat or improve a tissue condition. The compositions may be useful as space-filling agents, for example, in the form of scaffolds that provide augmentation, mechanical support, bulking, prevent tissue adhesions, or function as bioadhesives.

The phrase “tissue” refers to part of an organism consisting of an aggregate of cells having a similar structure and function. Examples include, but are not limited to, brain tissue, retina, skin tissue, hepatic tissue, pancreatic tissue, bone, nerve, cartilage, connective tissue, blood tissue, muscle tissue, cardiac tissue brain tissue, vascular tissue, renal tissue, pulmonary tissue, gonadal tissue, hematopoietic tissue and fat tissue. Preferably, the phrase “tissue” as used herein also encompasses the phrase “organ” which refers to a fully differentiated structural and functional unit in an animal that is specialized for some particular function. Non-limiting examples of organs include head, brain, eye, leg, hand, heart, liver kidney, lung, pancreas, ovary, testis, and stomach.

In one aspect, the present compositions may be utilized as scaffolding for soft tissue injuries such as, for example, injuries ranging from smooth muscle injuries, acute traumatic ruptures and lacerations to chronic overuse injuries, such as tendinitis. As an example, the use of the presently described compositions as biocompatible scaffolds may enhance tissue repair, for example, muscle or tendon healing and regeneration.

Scaffolds of this disclosure may initially fill a space otherwise occupied by natural tissue, and then provide a framework by which that tissue may be regenerated. For example, in some embodiments, the compositions are in the form of space filling agents or bulking agents for soft tissue and smooth muscle. According to another aspect of the invention there is provided a method of treating a subject having a disorder characterized by tissue damage or loss.

As used herein the phrase “disorder characterized by tissue damage or loss” refers to any disorder, disease or condition exhibiting a tissue damage (i.e., non-functioning tissue, cancerous or pre-cancerous tissue, broken tissue, fractured tissue, fibrotic tissue, or ischemic tissue) or a tissue loss (e.g., following a trauma, an infectious disease, a genetic disease, and the like) which require tissue regeneration. Examples for disorders or conditions requiring tissue regeneration include, but are not limited to, liver cirrhosis such as in hepatitis C patients (liver), Type-1 diabetes (pancreas), cystic fibrosis (lung, liver, pancreas), bone cancer (bone), burn and wound repair (skin), age related macular degeneration (retina), myocardial infarction, myocardial repair, CNS lesions (myelin), articular cartilage defects (chondrocytes), bladder degeneration, intestinal degeneration, and the like.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

When introduced into tissue, many of the present hydrogel compositions stimulate vessel generation, tissue in-growth and/or other new tissue generation into and adjacent the composition. In some embodiments, the compositions stimulate or encourage collagenesis of tissue.

Some embodiments include a method of generating tissue comprising contacting a tissue with a hydrogel composition to generate an additional amount of the tissue. Tissue types that may be generated include, but are not limited to, adipose tissue, muscle tissue, tendon tissue, cardiovascular tissue, neural tissue, bone tissue, and the like.

In one aspect, a hydrogel composition is provided which is injectable through a conventional sized cannula, for example, a cannula having gauge of about 12 gauge, about 25 gauge, 30 gauge, or finer, under normal manual pressure with a smooth extrusion plateau.

Crosslinked hyaluronic acid, collagen, and crosslinked collagen, such as those used in dermal fillers, generally do not promote cellular infiltration and tissue in-growth. Similarly, collagen simply blended into hyaluronic acid hydrogels generally does not promote tissue integration or de novo tissue generation. However, some compositions, for example, hydrogel compositions, described herein have been discovered to promote cellular migration, and tissue formation within the hydrogels, when implanted in vivo.

In one aspect, a hydrogel composition is provided which comprises water and a crosslinked macromolecular matrix. The crosslinked molecular matrix may comprise a hyaluronic acid component and a collagen component, wherein the hyaluronic acid component is crosslinked to the collagen component by a crosslinking component. A crosslinking component may comprise a plurality of crosslink units, wherein at least a portion of the crosslink units comprise an ester bond or an amide bond.

A crosslinked macromolecular matrix for a hydrogel may be synthesized by coupling a hyaluronic acid with a collagen using a coupling agent, such as a carbodiimide. In these hydrogels, hyaluronic acid may serve as a biocompatible water-binding component, providing bulk and isovolumetric degradation. Additionally, collagen may impart cell adhesion and signaling domains to promote cell attachment, migration, and other cell functions such as extra-cellular matrix deposition. The biopolymers form homogeneous hydrogels with tunable composition, swelling, and mechanical properties. Compositions can be made to be injectable for minimally invasive implantation through syringe and needle.

Hyaluronic acid is a non-sulfated glycosaminoglycan that enhances water retention and resists hydrostatic stresses. It is non-immunogenic and can be chemically modified in numerous fashions. Hyaluronic acid may be anionic at pH ranges around or above the pKa of its carboxylic acid groups.

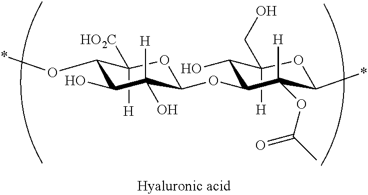

Collagen is a protein that forms fibrils and sheets that bear tensile loads. Collagen also has specific integrin-binding sites for cell adhesion and is known to promote cell attachment, migration, and proliferation. Collagen may be positively charged because of its high content of basic amino acid residues such as arginine, lysine, and hydroxylysine.

Because hyaluronic acid may be anionic and collagen may be cationic, the two macromolecules may form polyionic complexes in aqueous solution. A polyionic complex may be significantly less soluble in water than either hyaluronic acid or collagen, and thus may precipitate out of aqueous solution when the two macromolecules are together in a mixture.

Under certain conditions, a hyaluronic acid and a collagen may be combined in an aqueous liquid in which both components are soluble. A hyaluronic acid and a collagen may then be crosslinked while both are dissolved in an aqueous solution to form a hydrogel. Reaction conditions such as the concentration of hyaluronic acid, the concentration of collagen, the pH of the solution, and salt concentration may be adjusted to help to prevent polyionic complex formation between anionic hyaluronic acid and cationic collagen. They may also help to prevent collagen microfibril formation.

Some embodiments include a method of crosslinking hyaluronic acid and collagen. This method generally comprises a dissolution step which results in an aqueous pre-reaction solution. In a dissolution step, hyaluronic acid and collagen are dissolved in an aqueous solution that has a low pH and/or a salt to form an aqueous pre-reaction solution.

A hyaluronic acid-collagen crosslinking method further comprises an activation step. In an activation step, an aqueous pre-reaction solution is modified at least by adding a water soluble coupling agent and/or by increasing the pH of the solution. If needed, a salt may also be added to keep the hyaluronic acid and collagen in solution at the higher pH. Thus, a crosslinking reaction mixture comprises hyaluronic acid and collagen dissolved or dispersed in an aqueous medium, a water soluble coupling agent, and a salt, and has a higher pH than the aqueous pre-reaction solution from which it was derived. The crosslinking reaction mixture is allowed to react to thereby crosslink the hyaluronic acid and the collagen.

In some embodiments, the pH of the aqueous pre-reaction solution may be increased and a substantial amount of fiber formation may be allowed to occur in the solution before adding the water soluble coupling agent. In some embodiments, the water soluble coupling agent may be added to the aqueous pre-reaction solution before substantially any fiber formation occurs.

A crosslinking reaction mixture can react to form a crosslinked macromolecular matrix. Since reaction occurs in an aqueous solution, a crosslinked macromolecular matrix may be dispersed in an aqueous liquid in hydrogel form as it is formed by a crosslinking reaction. A crosslinked macromolecular matrix may be kept in hydrogel form because, in many instances, a crosslinked macromolecular matrix may be used in hydrogel form.

In some embodiments, an aqueous pre-reaction solution or a crosslinking reaction mixture may further comprise about 10% to about 90% of an organic solvent in which hyaluronic acid has poor solubility, such as ethanol, methanol, isopropanol, or the like.

After a crosslinking reaction has occurred, the crosslinked macromolecular matrix may be particulated or homogenized through a mesh. This may help to form an injectable slurry or hydrogel. A mesh used for particulating a crosslinked macromolecular matrix may have any suitable pore size depending upon the size of particles desired. For example, the mesh may have a pore size of about 10 microns to about 300 microns, for example, about 20 microns to about 100 microns. In some embodiments, the mesh size is about 50 microns, about 60 microns, about 70 microns, about 80 microns, or about 90 microns.

A hydrogel comprising a crosslinked molecular matrix may be treated by dialysis for sterilization or other purposes. Dialysis may be carried out by placing a semipermeable membrane between the hydrogel and another liquid so as to allow the hydrogel and the liquid to exchange molecules or salts that can pass between the membrane.

A dialysis membrane may have a molecular weight cutoff that may vary. For example, the cutoff may be about 5,000 daltons to about 100,000 daltons, about 10,000 daltons to about 30,000 daltons, or about 20,000 daltons.

The dialysis may be carried out against a buffer solution, or the liquid on the other side of the membrane from the hydrogel may be a buffer solution. In some embodiments, the buffer solution may be a sterile phosphate buffer solution that may comprise phosphate buffer, potassium chloride, and/or sodium chloride. A sterile phosphate buffer solution may be substantially isosmotic with respect to human physiological fluid. Thus, when dialysis is complete, the liquid component of a hydrogel may be substantially isosmotic with respect to human physiological fluid.

In some embodiments, a crosslinked macromolecular complex may further comprise an aqueous liquid. For example, the crosslinked macromolecular complex may absorb the aqueous liquid so that a hydrogel is formed. An aqueous liquid may comprise water with a salt dissolved in it, such as a phosphate buffer, sodium chloride, potassium chloride, etc. In some embodiments, an aqueous liquid may comprise water, sodium chloride at a concentration of about 100 mM to about 200 mM, potassium chloride at a concentration of about 2 mM to about 3 mM, and phosphate buffer at a concentration of about 5 mM to about 15 mM, wherein the pH of the liquid is about 7 to about 8.

In one aspect, the hydrogels are in the form of a product for treating or augmenting soft tissue, the product including an implantable composition comprising a hydrogel of a crosslinked macromolecular matrix.

In some embodiments, a hydrogel of a crosslinked macromolecular complex may have a storage modulus of about 1 Pa to about 10,000 Pa, about 50 Pa to 10,000 Pa, about 500 Pa to about 1000 Pa, about 556 Pa, about 560 Pa, about 850 Pa, about 852 Pa, or any value in a range bounded by, or between, any of these values.

In some embodiments, a hydrogel of a crosslinked macromolecular complex may have a loss modulus of about 1 Pa to about 500 Pa, about 10 Pa to 200 Pa, about 100 Pa to about 200 Pa, about 20 Pa, about 131 Pa, about 152 Pa, or any value in a range bounded by, or between, any of these values.

In some embodiments, a hydrogel of a crosslinked macromolecular complex may have an average extrusion force of about 20 N to 30 N, or about 25 N, when the hydrogel is forced through a 30 G needle syringe by moving the plunger of a 1 mL syringe containing the hydrogel at a rate of 100 mm/min for about 11 mm, and measuring the average force from about 4 mm to about 10 mm.

A crosslinked macromolecular complex may have tunable swelling properties based on reaction conditions and hydrogel dilution. In some embodiments, a crosslinked macromolecular complex may have a swelling ratio of about 1 to about 7. A swelling ratio is the ratio of the weight of the crosslinked macromolecular complex when saturated with water to the weight of the crosslinked macromolecular complex without any water. More specifically, the swelling ratio if the ratio of the mass of the gel which has been allowed to fully swell to the mass of the gel at its initial concentration.

In a crosslinking reaction, the molecular weight of a hyaluronic acid may vary. In some embodiments, a hyaluronic acid may have a molecular weight of about 300,000 daltons to about 10,000,000 daltons, for example, about 500,000 daltons to about 5,000,000 daltons, or about 1,000,000 daltons to about 3,000,000 daltons. When the crosslinking reaction occurs, the resulting crosslinked macromolecular product may have a hyaluronic acid component derived from the hyaluronic acid in the crosslinking reaction. Thus, the ranges recited above may also apply to the molecular weight of a hyaluronic acid component, e.g. about 300,000 daltons to about 10,000,000 daltons, about 500,000 daltons to about 5,000,000 daltons, or about 1,000,000 daltons to about 3,000,000 daltons. The term “molecular weight” is applied in this situation to a portion of the matrix even though the hyaluronic acid component may not actually be a separate molecule due to the crosslinking.

The concentration of hyaluronic acid in an aqueous pre-reaction solution or a crosslinking reaction mixture may vary. In some embodiments, hyaluronic acid is present at about 3 mg/mL to about 100 mg/mL, about 6 mg/mL to about 24 mg/mL, about 1 mg/mL to about 30 mg/mL, about 1.7 mg/mL, about 3 mg/mL, about 6 mg/mL, about 12 mg/mL, about 16 mg/mL, or about 24 mg/mL

Any type of collagen may be used in the methods and compositions described herein. In some embodiments, collagen type I, collagen type III, collagen type IV, collagen type VI, or a combination thereof, may be used. In some embodiments, a collagen or a collagen component comprises collagen type I or collagen type III. In some embodiments, the collagen component comprises collagen type V.

A collagen may be derived from cell culture, animal tissue, or recombinant means, and may be derived from human, porcine, or bovine sources. Some embodiments comprise collagen derived from human fibroblast culture. Some embodiments comprise collagen that has been denatured to gelatin. The source and/or collagen extraction/processing conditions can alter the way in which collagen macromolecules bundle together. These higher order structures can have effects on the gel physical properties (stiffness, viscosity) and may also have an effect on the reactivity of the collagen to crosslinking reagents.

Collagen concentration in an aqueous pre-reaction solution or a crosslinking reaction mixture may vary. In some embodiments, collagen may be present at a concentration of about 1 mg/mL to about 40 mg/mL, about 1 mg/mL to about 15 mg/mL, about 3 mg/mL to about 12 mg/mL, about 1.7 mg/mL, about 3 mg/mL, about 6 mg/mL, about 8 mg/mL, or about 12 mg/mL. The collagen concentration has an effect on the physical properties of the gel (stiffness, viscosity). In general, higher collagen concentrations lead to a higher elastic modulus.

In some embodiments, the weight ratio of hyaluronic acid to collagen in a aqueous pre-reaction solution or a aqueous pre-reaction solution or a crosslinking reaction mixture (e.g. [wt hyaluronic acid]/[wt collagen]) may be about 0.5 to about 3, about 1 to about 3, about 1 to about 2, about 1, or about 2. When the crosslinking reaction occurs, the resulting crosslinked macromolecular product may have a collagen component derived from the collagen in the crosslinking reaction. Thus, the resulting crosslinked macromolecular matrix may have a weight ratio of hyaluronic acid component to collagen component that corresponds to the weight ratio in the crosslinking reaction, e.g. about 0.5 to about 3, about 1 to about 3, about 1 to about 2, about 1, or about 2.

In other embodiments of the invention, the compositions have an HA to collagen weight ratio of between about 0.5 to 1 and about 7 to 1. For example, the weight ratio of hyaluronic acid to collagen may be about 1, about 2, about 3, about 4, about 5, about 6 or about 7. Specific examples are provided elsewhere herein.

A salt may help to screen the negative charges of hyaluronic acid from positive charges of collagen, and may thus prevent precipitation of a polyionic ion complex from solution. However, high concentrations of salt may reduce the solubility of some components in solution. Thus, in some embodiments, the salt concentration of an aqueous pre-reaction solution or a crosslinking reaction mixture may be high enough to screen the charges so that the polyionic ion complex is not formed, but also low enough so that the components of the mixture remain in solution. For example, the total salt concentration of some aqueous pre-reaction solutions or crosslinking reaction mixtures may be about 10 mM to about 1 M, for example, between about 5 mM to about 0.5 M, for example, between about 2 mM to about 0.2 M.

Some salts in an aqueous pre-reaction solution or a crosslinking reaction mixture may be non-coordinating buffers. Any non-coordinating buffer may be used that is capable of buffering the mixture and does not coordinate with metal atoms or ions in the collagen. In some embodiments, the buffer is a buffer which will not react with the crosslinking reagents (carbodiimide and additive). For example, in some embodiments, acetate or phosphate buffers are not used. Examples of suitable non-coordinating buffers may include, but are not limited to, 2-(N-morpholino)ethanesulfonic acid (MES), 3-(N-morpholino)propanesulfonic acid (MOPS), 4-(2-hydroxyethyl)-1-piperazinyl)ethanesulfonic acid (HEPES), 3-[4-(2-hydroxyethyl)-1-piperazinyl]propanesulfonic acid (HEPPS), N-cyclohexyl-2-aminoethanesulfonic acid (CHES), N-cyclohexyl-3-aminopropanesulfonic acid (CAPS), etc.

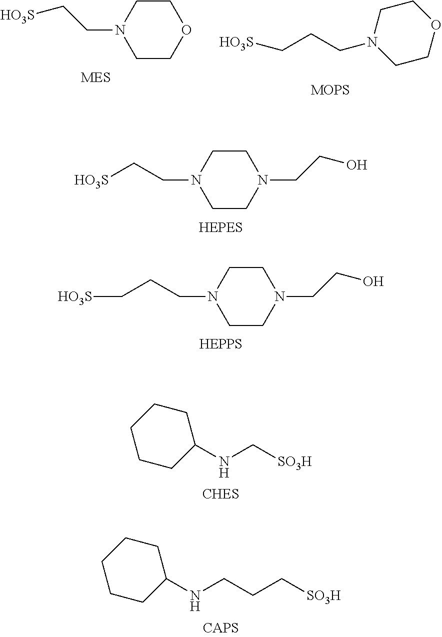

The concentration of a non-coordinating buffer may vary. For example, some aqueous pre-reaction solutions or crosslinking reaction mixtures may have a buffer concentration in a range of about 10 mM to about 1 M, about 10 mM to about 500 mM, about 20 mM to about 100 mM, or about 25 mM to about 250 mM. Some aqueous pre-reaction solutions or crosslinking reaction mixtures comprise MES at a concentration of about 20 mM to about 200 mM, about 20 mM to about 100 mM, about 100 mM, or about 180 mM.

Non-buffering salts may also be included in an aqueous pre-reaction solution or a crosslinking reaction mixture as an alternative to, or in addition, to buffering salts. Some examples may include sodium chloride, potassium chloride, potassium bromide, sodium bromide, lithium chloride, lithium bromide, sodium iodide, and potassium iodide. The concentration of a non-buffering salt may vary. For example, some mixtures may have a non-buffering salt concentration in a range of about 10 mM to about 1 mM, about 30 mM to about 500 mM, or about 50 mM to about 300 mM. In some embodiments, sodium chloride may be present at a concentration in a range of about 0.5% w/v to about 2% about 0.9% w/v, about 1.6% w/v, about 20 mM to about 1 mM, about 40 mM to about 500 mM, about 50 to 300 mM, about 80 mM to about 330 mM, about 150 mM, or about 270 mM.

The pH of an aqueous pre-reaction solution may be lower than the pH of a crosslinking reaction mixture. If the salt content of the aqueous pre-reaction solution is low, the pH may be lower to enhance solubility of the hyaluronic acid and the collagen. If the salt content is higher, the pH may be higher in the aqueous pre-reaction solution. In some embodiments, the pH of the aqueous pre-reaction mixture is about 1 to about 8, about 3 to about 8, about 4 to about 6, about 4.7 to about 7.4, or about 5.4. For low salt concentrations, the pH may be about 1 to about 4 or about 1 to about 3.

In some embodiments, pH may be adjusted to neutral to allow collagen gelation or fiber formation before adding a coupling agent.

In some embodiments, the pH may be adjusted to neutral immediately prior to, around the time of, or after adding a coupling agent, such that collagen gelation is reduced or does not substantially occur.

Any water-soluble coupling agent may be used that can crosslink hyaluronic acid to collagen. Some non-limiting examples of a coupling agent include carbodiimides such as N,N′-dicyclohexylcarbodiimide (DCC), N,N′-diisopropylcarbodiimide (DIC), or 1-ethyl-3-(3-dimethylaminopropyl)carbodiimide (EDC). Carbodiimide coupling agents may facilitate ester or amide bond formation without becoming part of the linkage. However, other coupling agents that become part of the crosslinking group may be used. The concentration of a coupling agent may vary. In some embodiments, a coupling agent may be present at about 2 mM to about 150 mM, about 2 mM to about 50 mM, about 20 mM to about 100 mM, or about 50 mM. In some embodiments, the coupling agent is EDC that is present at a concentration of about 20 mM to about 100 mM, about 2 mM to about 50 mM, or about 50 mM.

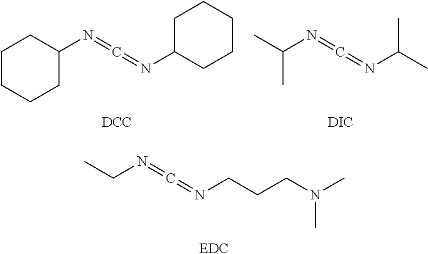

As a result of a crosslinking reaction, a crosslinked macromolecular matrix may comprise a crosslinking component that crosslinks or covalently connects the hyaluronic acid component to the collagen component. A crosslink component comprises a plurality of crosslink units, or individual covalent bonding links, between the hyaluronic acid component and the collagen component. At least a portion of the crosslink units comprise an ester bond or an amide bond. In some embodiments, at least a portion of the crosslink units may be —C(O)N— or —C(O)O—, where the N is a nitrogen from an amino acid residue.

An activating agent may be used to increase the ratio of amide bonds compared to ester bonds formed in the crosslinked product. In some embodiments, an activating agent may be a triazole such as hydroxybenzotriazole (HOBT) or 1-hydroxy-7-azabenzotriazole (HOAT); a fluorinated phenol such as pentafluorophenol; a succinimide such as N-hydroxysuccinimide (NHS) or N-hydroxysulfosuccinimide (sulfoNHS), and the like.

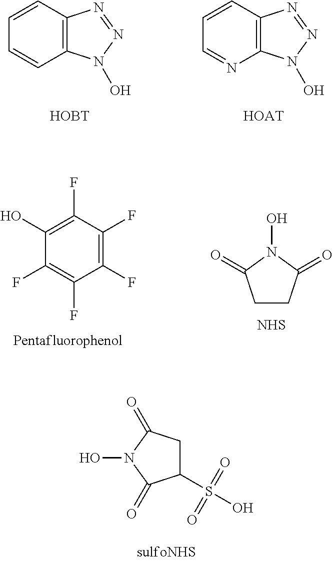

The concentration of an activating agent may vary. In some embodiments, the activating agent may have a concentration of about 2 mM to about 200 mM, about 2 mM to about 50 mM, about 20 mM to about 100 mM, or about 50 mM. In some embodiments, the activating agent may be NHS or sulfoNHS is at a concentration of about 2 mM to about 50 mM. In some embodiments, the activating agent may be N-hydroxysulfosuccinimide, sodium salt, at a concentration of about 20 mM to about 100 mM, or about 50 mM.

In some embodiments, a crosslinking reaction mixture may comprise a carbodiimide coupling agent and an activating agent. In some embodiments, the coupling agent is EDC and the activating agent is NHS or sulfoNHS. In some embodiments EDC is present at a concentration of about 2 mM to about 50 mM and NHS or sulfoNHS is present at about 2 mM to about 50 mM.

In some embodiments, a crosslinking reaction mixture may comprise hyaluronic acid at a concentration of about 1.7 mg/mL, collagen at a concentration of about 1.7 mg/mL, 2-(N-morpholino)ethanesulfonic acid at a concentration of about 100 mM, sodium chloride at a concentration of about 0.9 wt % or about 150 mM, 1-ethyl-3-(3-dimethylaminopropyl)carbodiimide at a concentration of about 50 mM, and N-hydroxysulfosuccinimide sodium salt at a concentration of about 50 Mm, wherein the solution has a pH of about 5.4.

In some embodiments, a crosslinking reaction mixture may comprise hyaluronic acid at a concentration of about 6 mg/mL, collagen at a concentration of about 6 mg/mL, 2-(N-morpholino)ethanesulfonic acid at a concentration of about 180 mM, sodium chloride at a concentration of about 1.6 wt % or about 270 mM, 1-ethyl-3-(3-dimethylaminopropyl)carbodiimide at a concentration of about 50 mM, and N-hydroxysulfosuccinimide sodium salt at a concentration of about 50 mM, wherein the solution has a pH of about 5.4.

In some embodiments, a crosslinking reaction mixture may comprise hyaluronic acid at a concentration of about 16 mg/mL of, collagen at a concentration of about 8 mg/mL, 2-(N-morpholino)ethanesulfonic acid at a concentration of about 100 mM, sodium chloride at a concentration of about 0.9 wt % or about 150 mM, 1-ethyl-3-(3-dimethylaminopropyl)carbodiimide at a concentration of about 50 mM, and N-hydroxysulfosuccinimide sodium salt at a concentration of about 50 mM, wherein the solution has a pH of between about 4.5 and 5.5, for example, about 5.2.

In some embodiments, a crosslinking reaction mixture may comprise hyaluronic acid at a concentration of about 12 mg/mL, collagen at a concentration of about 12 mg/mL, 2-(N-morpholino)ethanesulfonic acid at a concentration of about 100 mM, sodium chloride at a concentration of about 0.9 wt % or about 150 mM, 1-ethyl-3-(3-dimethylaminopropyl)carbodiimide at a concentration of about 50 mM, and N-hydroxysulfosuccinimide sodium salt at a concentration of about 50 mM.

In some embodiments, a crosslinking reaction mixture may comprise hyaluronic acid at a concentration of about 3 mg/mL, collagen at a concentration of about 3 mg/mL, 2-(N-morpholino)ethanesulfonic acid at a concentration of about 100 mM, sodium chloride at a concentration of about 0.9 wt % or about 150 mM, 1-ethyl-3-(3-dimethylaminopropyl)carbodiimide at a concentration of about 50 mM, and N-hydroxysulfosuccinimide sodium salt at a concentration of about 50 mM, wherein the solution has a pH of about 5.4.

In some embodiments, a crosslinking reaction mixture may comprise hyaluronic acid at a concentration of about 12 mg/mL, collagen at a concentration of about 6 mg/mL, 2-(N-morpholino)ethanesulfonic acid at a concentration of about 100 mM, sodium chloride at a concentration of about 0.9 wt % or about 150 mM, 1-ethyl-3-(3-dimethylaminopropyl)carbodiimide at a concentration of about 50 mM, and N-hydroxysulfosuccinimide sodium salt at a concentration of about 50 mM, wherein the solution has a pH of about 5.4.

In some embodiments, a crosslinking reaction mixture may comprise hyaluronic acid at a concentration of about 24 mg/mL, collagen at a concentration of about 12 mg/mL, 2-(N-morpholino)ethanesulfonic acid at a concentration of about 100 mM, sodium chloride at a concentration of about 0.9 wt % or about 150 mM, 1-ethyl-3-(3-dimethylaminopropyl)carbodiimide at a concentration of about 50 mM, and N-hydroxysulfosuccinimide sodium salt at a concentration of about 50 mM, wherein the solution has a pH of about 5.4.

In some embodiments, a crosslinking reaction mixture may comprise hyaluronic acid at a concentration of about 1 mg/mL to about 20 mg/mL, collagen at a concentration of about 1 mg/mL to about 15 mg/mL, 2-(N-morpholino)ethanesulfonic acid at a concentration of about 20 mM to about 200 mM, sodium chloride at a concentration of about 0.5 wt % to about 2 wt % or about 80 mM to about 330 mM, 1-ethyl-3-(3-dimethylaminopropyl)carbodiimide at a concentration of about 20 mM to about 100 mM, and N-hydroxysulfosuccinimide sodium salt at a concentration of about 20 mM to about 100 mM, wherein the solution has a pH of about 4 to about 6.

### Example 1

### Method of Making a HA/Collagen Hydrogel Composition

Solutions of hyaluronic acid (HA) and collagen were produced by dissolving 15 mg of 2.0 MDa hyaluronic acid in 5 mL of human collagen(III) solution at 3 mg/mL in 0.01 N hydrochloric acid (Fibrogen). The hyaluronic acid/collagen solution was then lyophilized at −50° C. and 0.02 Torr. The resulting sponges were soaked in 20 mL of ethanol:water mixture at ratios varying from 1:2 to 5:1 with 50 mM of 1-ethyl-3-(3-dimethylaminopropyl)carbodiimide and 50 mM of N-hydroxysulfosuccinimide sodium salt for 24 hrs. The crosslinked gels were then washed in 70% isopropanol/30% water for sterilization followed by five washes in sterile phosphate buffer for purification.

### Example 2

### Method of Making a HA/Collagen Hydrogel Composition

A solution of HA at 3.4 mg/mL was created by dissolving 34 mg of 2 MDa HA in 10 mL of 100 mM MES buffer with 0.9 wt % NaCl, pH 4.7. Upon full hydration and dissolution of the HA, this solution was mixed with 10 mL of 3.4 mg/mL human collagen(III) solution in 100 mM HCl. The pH of the resulting HA/collagen(III) solution was adjusted to 5.4 with 10 mM NaOH solution. EDC (192 mg) and 217 mg of sulfoNHS (50 mM each) were added to the HA/collagen(III) solution and mixed thoroughly. The crosslinking reaction proceeded for 18 hrs before the gel was particulated through a 100 micron pore-sized mesh.

### Example 3

### Method of Making a HA/Collagen Hydrogel Composition

Rat tail collagen(I) in 0.01 N hydrochloric acid (Invitrogen) was concentrated from 5 mg/mL to 8 mg/mL using a centrifugal filtration device with 20 kDa molecular weight cutoff. HA (160 mg, 2 MDa) was added to 10 mL of the collagen solution and allowed to hydrate for 60 minutes. The solution was then homogenized by passing from syringe to syringe through a luer-luer connector. NaCl (93 mg) and 201 mg of MES were added to the solution and mixed. EDC (98 mg) and 111 mg of sulfoNHS were added to the solution and quickly mixed. Finally, 200 μL of 1 N NaOH was added to the solution which was mixed by syringe-to-syringe passing. The reaction solution was transferred to a glass vial and centrifuged for 5 min at 4000 RPM to remove air bubbles. The gel was then particulated through a 60 micron pore-sized mesh. Following sizing, the gel was sterilized by dialysis through a 20 kDa molecular-weight cut-off cellulose ester membrane against 70% isopropanol/30% water for 3 hrs at 4° C. Dialysis was then continued against sterile phosphate buffer for 48 hrs at 4° C. with three changes of buffer. The gel was then dispensed into syringes under aseptic conditions.

### Example 4

### Method of Making a HA/Collagen Hydrogel Composition

Rat tail collagen(I) in 0.01 N hydrochloric acid (Invitrogen) was concentrated from 5 mg/mL to 12 mg/mL using a centrifugal filtration device with 20 kDa molecular weight cutoff. HA (120 mg, 2 MDa) was added to 10 mL of the collagen solution and allowed to hydrate for 60 minutes. The solution was then homogenized by passing from syringe to syringe through a luer-luer connector. NaCl (93 mg) and 201 mg of MES were added to the solution and mixed. EDC (98 mg) and 111 mg of sulfoNHS were added to the solution and quickly mixed. Finally, 200 μL of 1 N NaOH was added to the solution which was mixed by syringe-to-syringe passing. The reaction solution was transferred to a glass vial and centrifuged for 5 min at 4000 RPM to remove air bubbles. The gel was then particulated through a 60 micron pore-sized mesh. Following sizing, the gel was sterilized by dialysis through a 20 kDa molecular-weight cut-off cellulose ester membrane against 70% isopropanol/30% water for 3 hrs at 4° C. Dialysis was then continued against sterile phosphate buffer for 48 hrs at 4° C. with three changes of buffer. The gel was then dispensed into syringes under aseptic conditions.

### Example 5

### Method of Making a HA/Collagen Hydrogel Composition

Rat tail collagen(I) in 0.01 N hydrochloric acid (Invitrogen) was concentrated from 5 mg/mL to 12 mg/mL using a centrifugal filtration device with 20 kDa molecular weight cutoff. HA (120 mg, 2 MDa) was added to 10 mL of the collagen solution and allowed to hydrate for 60 minutes. The solution was then homogenized by passing from syringe to syringe through a luer-luer connector. NaCl (93 mg), 201 mg of MES, and 200 μL of 1 N NaOH were added to the solution, mixed, and given 45 minutes for collagen polymerization. EDC (98 mg) and 111 mg of sulfoNHS were then added and the final solution was mixed by syringe-to-syringe passing. The reaction solution was transferred to a glass vial and centrifuged for 5 min at 4000 RPM to remove air bubbles. The gel was then particulated through a 60 micron pore-sized mesh. Following sizing, the gel was sterilized by dialysis through a 20 kDa molecular-weight cut-off cellulose ester membrane against 70% isopropanol/30% water for 3 hrs at 4° C. Dialysis was then continued against sterile phosphate buffer for 48 hrs at 4° C. with three changes of buffer. The gel was then dispensed into syringes under aseptic conditions.

### Example 6

### Rheology Characterization of the Compositions

Oscillatory parallel plate rheology was used to characterize the mechanical properties of gels using an Anton Paar MCR 301. A plate diameter of 25 mm was used at a gap height of 1 mm. A frequency sweep from 0.1 to 10 Hz at a fixed strain of 2% with logarithmic increase in frequency was applied followed by a strain sweep between 0.1% and 300% at a fixed frequency of 5 Hz with logarithmic increase in strain. The storage modulus (G′) and loss modulus (G″) were determined from frequency sweep measurements at 5 Hz.

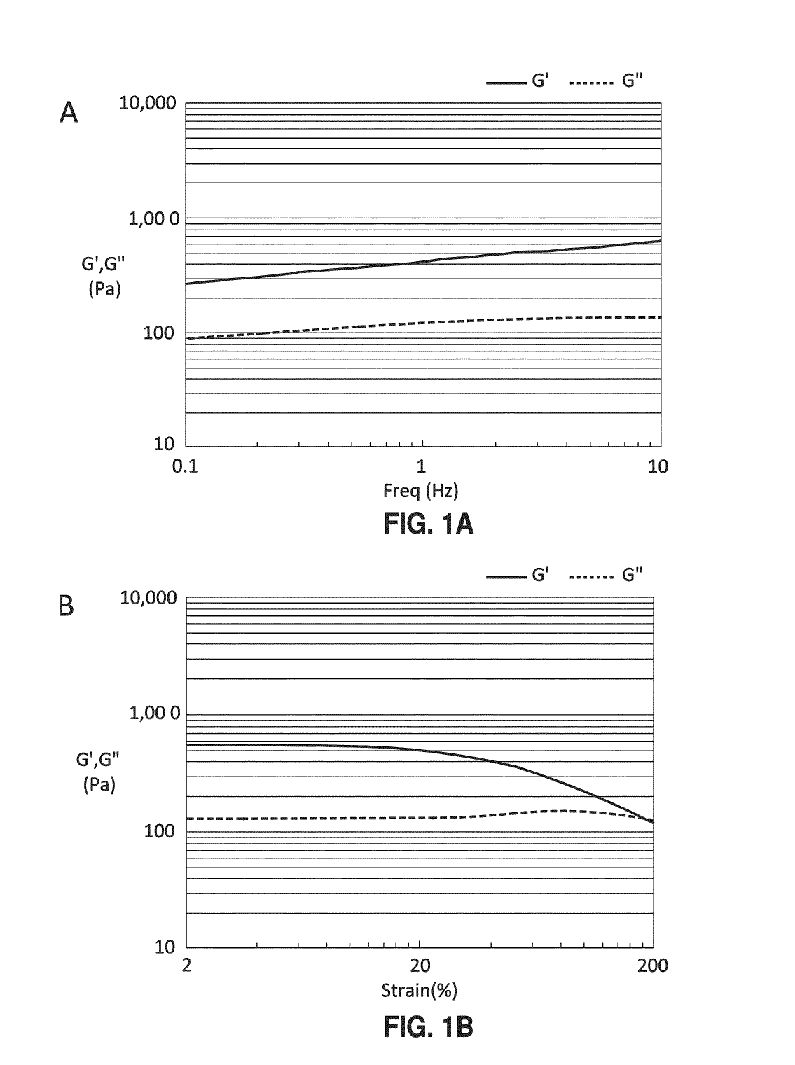

The gel from Example 4 had a storage modulus (G′) of 556 Pa and loss modulus (G″) of 131 Pa. The frequency sweep (A) and strain sweep (B) are shown in FIG. 1.

### Example 7

### Extrusion Test

In order to determine the force required to extrude the gels, they were ejected from 1 mL BD syringes through 30 G needles using an Instron 5564 with Bluehill 2 software. The plunger was pushed at a rate of 100 mm/min for 11.35 mm and the extrusion profile was recorded.

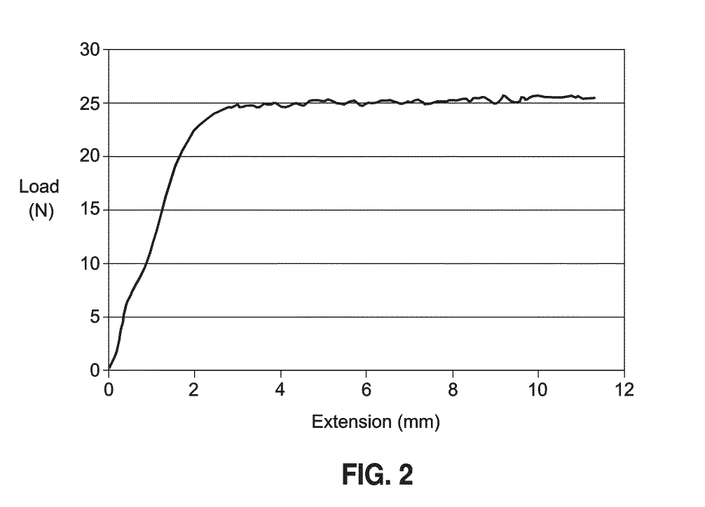

The extrusion profile through a 30 G needle for gel from Example 4 is shown in FIG. 2. The gel had an average extrusion force of 25 N from 4 through 10 mm.

### Example 8

### Method of Making HA/COLLAGEN Compositions

Hyaluronic acid, 2 MDa molecular weight, was dissolved in human collagen(I) solution in 0.01 N hydrochloric acid (Advanced BioMatrix). Sodium chloride was added at 0.9 wt % and 2-(N-morpholino)ethanesulfonic acid was added at 100 mM to the solution and mixed. The hyaluronic acid was allowed to hydrate for 1 hr and the solution was homogenized by syringe-to-syringe mixing. The pH of the solution was adjusted to 5.4 by addition of 1 N sodium hydroxide. 1-Ethyl-3-(3-dimethylaminopropyl) carbodiimide (50 mM) and N-hydroxysulfosuccinimide sodium salt (50 mM) were added to the hyaluronic acid/collagen solution and quickly mixed by syringe-to-syringe transfer. The solution was transferred to a glass vial and centrifuged for 5 min at 4000 RPM to remove air bubbles. The resulting gel was allowed to react for 16 hrs at 4° C. The gel was then particulated through a 100 micron pore-sized mesh. Following sizing, the gel was sterilized by dialysis through a 20 kDa molecular-weight cut-off cellulose ester membrane against 70% isopropanol/30% water for 3 hrs at 4° C. Dialysis was then continued against sterile phosphate buffer, pH 7.4, for 48 hrs at 4° C. with four changes of buffer. The gel was then dispensed into syringes under aseptic conditions.

This procedure was used to produce hydrogels with varying concentrations of hyaluronic acid and collagen. When required, human collagen(I) in 0.01 N hydrochloric acid was concentrated from 3 mg/mL to the desired reaction concentration in 20 kDa molecular-weight cut-off centrifugal filtration devices. A 50 mL sample of each gel was synthesized, sterilized by exposure to 70% isopropanol, and purified by dialysis against phosphate buffer, pH 7.4. The gels synthesized are described in Table 1 along with their rheological properties.

TABLE 1
Hyaluronic acid-human collagen(I) hydrogel synthesis
concentrations and rheological properties

| Sample | [HA] | [Col(I)] | G′ | G″ |
|---|---|---|---|---|
| ID | (mg/mL) | (mg/mL) | (Pa) | (Pa) |
| A | 3 | 3 | 199 | 24.6 |
| B | 12 | 6 | 1260 | 154 |
| C | 16 | 8 | 2450 | 288 |
| D | 12 | 12 | 3160 | 420 |
| E | 24 | 12 | 5440 | 433 |
| F | 12 | 3 | 1110 | 52.2 |
| G | 16 | 3 | 1490 | 60.6 |
| H | 20 | 3 | 1770 | 49.5 |

### Example 9

### Biopolymer Concentration of the Compositions

In order to determine the biopolymer concentration in gels, the weight of the hydrated gel was compared to that of dried gel. A 2 mL sample of gel was weighed and dried by flash-freezing in liquid nitrogen followed by lyophilization at −50° C. and 0.02 Torr. A solution of the appropriate buffer was also weighed and dried in the same fashion to account for salt content of the gel. The total solids content of the gel was calculated by dividing the dry weight by the wet volume, assuming 1 g/mL density for the wet gel, to give a value in mg/mL. The salt solids content was then subtracted from this value to determine the biopolymer concentration in the gel.

TABLE 2
Final concentrations of hyaluronic acid-human collagen(I) hydrogels

| | | | Final |
|---|---|---|---|
| Sample | | [Col(I)] | concentration |
| ID | [HA](mg/mL) | (mg/mL) | (mg/mL) |
| A | 3 | 3 | 5.3 |
| B | 12 | 6 | 16.3 |
| C | 16 | 8 | 19.4 |
| D | 12 | 12 | 22.6 |
| E | 24 | 12 | 31.6 |

### Example 10

### Swelling Ratios

Swelling ratios relative to initial water content were determined for gels by increase in weight when equilibrated with phosphate buffer. For each gel, approximately 1 mL was injected into a 15 mL Falcon tube and weighed, followed by addition of 10 mL of phosphate buffered saline, pH 7.4. The gels were thoroughly mixed with the buffer and vortexed for 30 seconds. The gels were then allowed to equilibrate in the buffer for 48 hrs at 4° C. After this time, the suspensions were centrifuged at 4000 RPM in a swinging bucket rotor for 5 minutes. The supernatant buffer was then decanted and the weight of the swollen gel was measured. The swelling ratio was determined by dividing the final weight of the swollen gel by the weight of the initial gel.

TABLE 3
Swelling ratios of hyaluronic acid-human collagen(I) hydrogels

| Sample | [HA] | [Col(I)] | Swelling |
|---|---|---|---|
| ID | (mg/mL) | (mg/mL) | ratio |
| A | 3 | 3 | 0.96 |
| B | 12 | 6 | 1.67 |
| C | 16 | 8 | 1.69 |
| D | 12 | 12 | 1.49 |
| E | 24 | 12 | 1.65 |

### Example 11

### Tissue Infiltration into the Compositions

Samples of hyaluronic acid-collagen(I) from Example 8 were implanted in a nude mouse model for evaluation of gel duration, angiogenic potential, and cellular infiltration. Gels were implanted as a bolus subcutaneously on the dorsum of female 6-week-old nude mice under anesthesia with two injections per mouse. One mL of each gel was implanted through a small incision by 16 G cannula and the incision closed using surgical glue. A total of 14 injections of each material were made. Syringes were weighed before and after injection to determine the weight of injected material. After 6 weeks, the gels were harvested and weight and volume (using liquid displacement) were determined for each sample. Samples were also processed for histology by hematoxylin and eosin (H&E) staining.

Weight and volume retention increased with total biopolymer concentration as shown in the table below. Samples with hyaluronic acid concentration of 24 mg/mL and collagen(I) concentration of 12 mg/mL had weight and volume retention greater than 100%, presumably from tissue infiltration into the gel and/or new tissue formation.

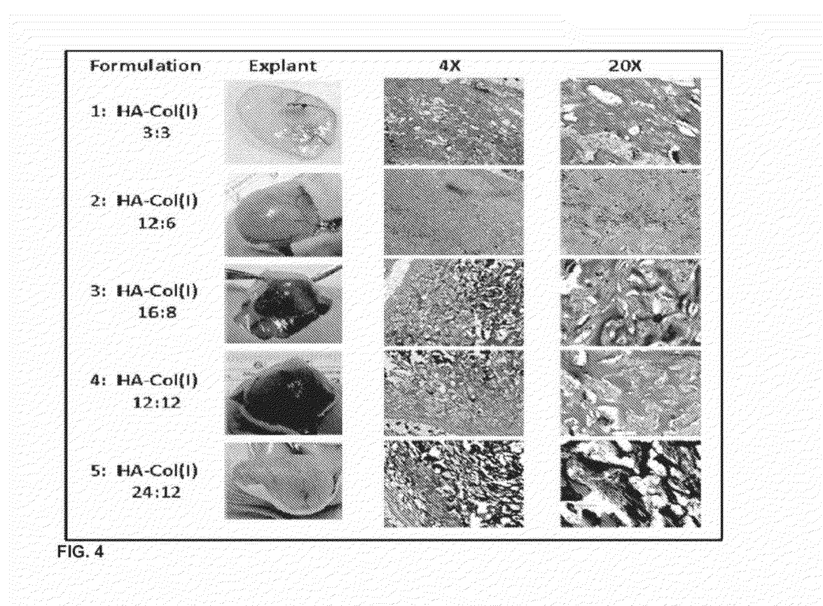

Photographs of gel explants along with 4× and 20× H&E micrographs are shown in FIG. 4. Gross examination of explanted gel samples indicated signs of neovascularization with blood vessels penetrating into the gels. Samples with increasing concentration had increased vascularization. Histology indicated cell and tissue infiltration into the gel implants. Samples A and B had new tissue deposition throughout the gel. Samples C, D and E had dense tissue infiltration within a zone at the edges of the gel.

Weight and volume retention (group means±standard deviation) of gels at 6 weeks:

| | [HA] | | | |
|---|---|---|---|---|
| Example | (mg/mL) | [Col(I)] (mg/mL) | weight (%) | volume (%) |
| A | 3 | 3 | 40.9 ± 4 | 38.4 ± 5 |
| B | 12 | 6 | 78.1 ± 15 | 81.2 ± 17 |
| C | 16 | 8 | 97.7 ± 3 | 93.2 ± 5 |
| D | 12 | 12 | 100.2 ± 7 | 104.8 ± 9 |
| E | 24 | 12 | 117.8 ± 5 | 118.2 ± 5 |

### Example 12

### HA/Collagen for Deep Tissue Filling after Moh's Surgery

This example illustrates the use of compositions and methods disclosed herein for repairing a facial deformity.

A 43-year-old woman presents with a significant facial depression and facial asymmetry, resulting from the removal of cancerous tissue from Moh's surgery that had been used a year ago to successfully treat skin cancer above her left cheekbone. Pre-operative evaluation of the patient includes routine history and physical examination in addition to thorough informed consent disclosing all relevant risks and benefits of the procedure. The physician evaluating the patient determines that she is a candidate for administration of the compositions and methods disclosed herein to smooth out the depression and restore symmetry to her face.

A HA/Collagen hydrogel composition as described herein and having a total HA concentration of about 12 mg/ml and a total collagen concentration of about 6 mg/ml, is provided in a 20 mL syringe. One-holed blunt infiltration cannulas (3 mm inner diameter) are used to place about 20 mL of the composition into deep tissue below the depression. The woman is monitored for approximately 60 days. The physician evaluates the treatment area and determines that the treatment was successful. The woman is happy with her restored appearance.

### Example 13

### HA/Collagen Compositions as a Space-Filling Scaffold (for Urinary Incontinence)

This example illustrates the use of compositions and methods disclosed herein for a treatment of urinary incontinence.

A 64-year-old woman presents with stress urinary incontinence (SUI) due to intrinsic sphincter deficiency. The physician determines that she is a candidate for a urethral bulking procedure which will inflate the submucosal tissues of the bladder neck. This procedures involves injecting a bulking agent into the wall of the urethra to provide bulk to up the bladder neck to effectively restore the mucosal seal mechanism of continence.

Pre-operative evaluation of the person includes routine history and physical examination in addition to thorough informed consent disclosing all relevant risks and benefits of the procedure. The physician evaluating the individual determines that she is a candidate for administration of the HA/collagen compositions and methods disclosed herein.

A HA/collagen hydrogel composition, such as made as described in the Examples herein, is provided in a syringe. The physician implants the composition into the urethra utilizing a standard, conventional urethral bulking procedure. By augmenting the urethral wall, the implanted composition increases urethral resistance to urinary flow. This is a minimally invasive procedure.

Every two weeks, the patient returns to the physician for additional treatments, identical to the first treatment, until the patient has had four total treatments.

The patient is monitored for approximately 30 days following the last treatment. Both the patient and her physician are satisfied with the results of the procedure because the woman has not suffered from any bouts of urinary incontinence since her last treatment. Approximately one year after the procedure, the woman indicates that her quality of life has improved.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the,” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A hydrogel composition useful for treating a human or veterinary subject having a disorder characterized by tissue damage or loss, the composition comprising:
a hyaluronic acid component; and
a collagen component;
wherein the hyaluronic acid component is crosslinked to the collagen component by an ester bond or an amide bond.

2. The composition of claim 1 having a weight ratio of the hyaluronic acid component to the collagen component of about 1 to about 7.

3. The composition of claim 1 having a weight ratio of the hyaluronic acid component to the collagen component of about 2.

4. The composition of claim 1 having a weight ratio of the hyaluronic acid component to the collagen component of about 3.

5. The composition of claim 1 having a weight ratio of the hyaluronic acid component to the collagen component of about 4.

6. The composition of claim 1 having a weight ratio of the hyaluronic acid component to the collagen component of about 6.

7. The composition of claim 1 having a collagen concentration of at least about 3 mg/ml to about 12 mg/ml.

8. The composition of claim 7 having a hyaluronic acid concentration of about 3 mg/ml to about 24 mg/ml.

9. The composition of claim 1 having a collagen concentration of about 3 mg/mL.

10. The composition of claim 9 having a hyaluronic acid concentration of about 3 mg/mL to about 20 mg/mL.

11. The composition of claim 9 having a hyaluronic acid concentration of about 3 mg/mL.

12. The composition of claim 9 having a hyaluronic acid concentration of about 12 mg/mL.

13. The composition of claim 9 having a hyaluronic acid concentration of about 20 mg/mL.

14. The composition of claim 1 having a collagen concentration of about 6 mg/mL.

15. The composition of claim 14 having a hyaluronic acid concentration of about 12 mg/mL.

16. The composition of claim 1 having a collagen concentration of about 8 mg/mL.

17. The composition of claim 16 having a hyaluronic acid concentration of about 16 mg/mL.

18. The composition of claim 1 having a collagen concentration of about 12 mg/mL.

19. The composition of claim 18 having a hyaluronic acid concentration of about 12 mg/mL to about 24 mg/mL.

20. The composition of claim 18 having a hyaluronic acid concentration of about 12 mg/mL.

21. The composition of claim 18 having a hyaluronic acid concentration of about 24 mg/mL.

22. A method for treating a human or veterinary subject having a disorder characterized by soft tissue damage or loss, the method comprising:
introducing into soft tissue in a patient, a hydrogel composition wherein the hydrogel composition comprises water and a crosslinked macromolecular matrix comprising
a hyaluronic acid component, and
a collagen component,
wherein the hyaluronic acid component is crosslinked to the collagen component by a crosslinking component, and
wherein the crosslinking component comprises a plurality of crosslink units, wherein at least a portion of the crosslink units comprise an ester bond or an amide bond.

23. The method of claim 22 wherein the hydrogel composition has a weight ratio of the hyaluronic acid component to the collagen component of about 1 to about 7.

24. The method of claim 22 wherein the hydrogel composition has a collagen concentration of about 3 mg/mL to about 12 mg/mL.

25. The method of claim 24 wherein the hydrogel composition has a hyaluronic acid concentration of about 3 mg/mL to about 24 mg/mL.

26. The method of claim 22 wherein the hydrogel composition has a collagen concentration of about 3 mg/mL and a hyaluronic acid concentration of about 3 mg/mL.

27. The method of claim 22 wherein the hydrogel composition has a collagen concentration of about 3 mg/mL and a hyaluronic acid concentration of about 12 mg/mL.

28. The method of claim 22 wherein the hydrogel composition has a collagen concentration of about 3 mg/mL and a hyaluronic acid concentration of about 16 mg/mL.

29. The method of claim 22 wherein the hydrogel composition has a collagen concentration of about 3 mg/mL and a hyaluronic acid concentration of about 20 mg/mL.
